(19) United States
(12) Patent Application Publication
(10) Pub. No.: US 2010/0099171 A1
(43) Pub. Date: Apr. 22, 2010

(54) VITRIFICATION APPARATUS FOR MICRODROP VITRIFICATION OF CELLS AND A METHOD OF MICRODROP VITRIFICATION OF CELLS USING THE APPARATUS

(76) Inventors: Jang-Chi Huang, Herng Chuen Chen (TW); Hsin-Hung Lin, Herng Chuen Chen (TW)

Correspondence Address: Hershkovitz & Associates, LLC, 2845 Duke Street, Alexandria, VA, 22314, US

(21) Appl. No.: 12/647,387

(22) Filed: Dec. 24, 2009

Related U.S. Application Data
(62) Division of application No. 11/203,311, filed on Aug. 15, 2005.

(51) Int. Cl. C12M 1/00

(52) U.S. Cl. 435/283.1

(57) ABSTRACT

An apparatus for microdrop vitrification has a tank and two wings. The tank has a bottom, two side edges, two top edges, two sidewalls, a lowest bottom, a drain and a spout. The drain is defined in one of the sidewalls at the bottom of the tank. The spout is mounted on the bottom of the tank and is connected to the drain. The wings are integrally formed respectively with and extend out from the top edges. The present invention also relates to a microdrop forming device, a method for microdrop vitrification and a method for recovering vitrified cells.

### RELATED PATENT APPLICATION

This application is a divisional of U.S. Non-Provisional patent application Ser. No. 11/203,311 filed on Aug. 15, 2005. The entire contents of which are incorporated herein by reference.

### BACKGROUND OF THE INVENTION

1. Field of the Invention

The present invention relates to an apparatus for microdrop vitrification of cells and also relates to a method for microdrop vitrification of cells using the apparatus.

2. Description of Related Art

A conventional method for freezing cells includes a traditional slow freezing method. However, the traditional slow freezing method is time consuming, and an expensive apparatus is needed. To increase freezing rates and devise an easy freezing procedure, a vitrification technique has been developed. In 1989, the vitrification technique was developed for mouse embryos. However, the freezing rate and the survival rate are still low. In 1997, Vajta et al. (1997) first vitrified bovine embryos in straws with the open pulled straw (OPS) method. The diameter of each straw is half the diameter of a conventional 0.25 ml straw. Successful cryopreservation of bovine embryos with the OPS method increased the cooling rate by 10 times to approximately 25,000° C./min. Such results assisted the development of the OPS methods. The advantage of the OPS vitrification method is the rapid cooling rate. However, the disadvantages of the OPS method include having to pull the straws by hand, the diameter of each pulled straw not being identical and requiring specially trained technicians with particular skills in techniques to perform the OPS vitrification method. Many apparatuses and methods have been used in an attempt to improve the cooling rate and to simplify the freezing procedure, such as EM grids, cryoloops, nylon mesh, droplets and solid surface vitrification.

Increasing the cooling rate is one of the key factors in successful vitrification. In addition to the size of a container to hold the cells to be vitrified, the shape and the material of a container also affect the cooling rate. Most of developed vitrification methods need containers or carriers to hold the desired cells and freezing medium. However, the containers act as insulators and impede the achievement of low temperatures. The containers can cause an immediate vaporization while they are plunging into the liquid nitrogen from the room temperature, hence to elevate the ambient temperature of the freezing object. Furthermore, the vaporized nitrogen forms a warmer vapor barrier between the container holding the desired cells and the liquid nitrogen and directly impedes the transfer of heat from the desired cells to the liquid nitrogen. The volume of the medium carried the cells for vitrification is one of the factors that affect the cooling rate as well. A 1 to 2 μl of carrying medium has been thought to be optimal for improving the cooling rate during vitrification (Liebermann et al., 2002). However, to generate a 1 or 2 μl droplet from a pipette or a capillary without having the capillary being pretreated is somewhat difficult. Accordingly, the volume sizes of droplets vitrified directly in liquid nitrogen or on solid surface partially immerging in liquid nitrogen were larger than 3 μl (Arav & Zeron, 1997; Atabay et al., 2004), or roughly 1 to 2 μl (Dinnyes et al., 2000).

Cryoprotectants, such as ethylene glycol, polyethylene glycol, dimethylsulfoxide, glycerol, propanediol, sugars, methyl pentanediol, and others well known in the art, can be toxic to sensitive cells such as oocytes and embryos when used in large dosages during cryopreservation.

Currently, the vitrification methods that have been developed are still needed to be modified to increase the cooling rate, to improve the viability of frozen-thawed animal cells and to simplify the applied apparatus and the manipulation procedures. The current invention provides a vitrification technique with high repeatability and high survival rate of frozen-thawed animal cells and is particularly useful for freezing embryos.

### SUMMARY OF THE INVENTION

The primary objective of the present invention is to provide a simple vitrification apparatus and a microdrop-forming device to simplify the methods of forming, vitrifying and thawing microdrops and improve the survival rate of vitrified cells.

The present invention relates to an apparatus for microdrop vitrification, a microdrop forming device, a method for microdrop vitrification of cells and a method of recovering vitrified cells.

The vitrification apparatus being used to vitrify and to recover microdrops, is mounted on a container of liquid nitrogen and comprises a tank and two wings.

The tank is partially submerged in the liquid nitrogen and has a bottom, two top edges, two sidewalls, a lowest bottom, a drain and a spout.

The drain is defined in one of the sidewalls at the bottom of the tank.

The spout is mounted on the bottom of the tank and is connected to the drain.

The wings are integrally formed respectively with and extend out from the top edges.

The microdrop-forming device comprises a modified capillary, a silicon tube and a micro-syringe. The modified capillary has an inner surface coating with a silicon membrane by siliconization, a calibrated outer surface, a capillary tip and a proximal end.

The silicon membrane is formed on the inner surface.

The method for microdrop vitrification of cells comprises providing cells, culturing the cells, forming a microdrop, dropping the microdrop into liquid nitrogen to form glass-like beads, collecting and storing the beads. The cells are cultured in a culture medium containing a cryoprotectant for a short period. The microdrop with desired volume size is formed correctly and uniformly from the microdrop-forming device as previously described. The microdrop is then dropped into the liquid nitrogen in the tank of the vitrification apparatus. The glass-like beads are recovered and collected in a vial mounted on the spout. The glass-like beads are stored at cryogenic temperatures.

The method for recovering cells comprises mounting the vitrification apparatus with a filter on a container, pouring the vitrified microdrops into the tank, recovering and placing the glass-like beads in a culture medium to thaw.

Further benefits and advantages of the present invention will become apparent after a careful reading of the detailed description.

### BRIEF DESCRIPTION OF THE DRAWINGS

FIG. 1 is an exploded perspective view of a vitrification apparatus for microdrop vitrification in accordance with the present invention;

FIG. 2 is a perspective view of the vitrification apparatus for microdrop vitrification in FIG. 1 on a container containing liquid nitrogen;

FIG. 3 is a perspective view of the vitrification apparatus for microdrop vitrification in FIG. 1 with a vial mounted on the apparatus;

FIG. 4 is an exploded perspective view of the vitrification apparatus for microdrop vitrification in FIG. 1 when microdrops are collected; and

FIG. 5 is a side view in partial section of a microdrop forming device.

### DETAILED DESCRIPTION OF THE INVENTION

The present invention relates to a vitrification apparatus for microdrop vitrification, a microdrop-forming device, a method for vitrification of cells in a microdrop and a method for recovering vitrified microdrops. The microdrop is formed from the microdrop-forming device and has a uniform volume as small as 1 or 2 μl, so the microdrop can be vitrified as quick as possible while it is dropped into liquid nitrogen within the invented apparatus. The volume of each microdrop is in the range of 1 to 15 μl, preferably in the range of 5 to 9 μl, more preferably in the range of 3 to 4 μl and most preferably in the range of 1 to 2 μl. Each microdrop contains from 1 to 20 cells, preferably 5 to 9 cells and more preferably 2 to 4 cells. The cells may be from microorganisms, plants or animals. The cells from animals are preferably oocytes or embryos, and the embryos are preferably at the blastocyst stage.

The following definitions are provided to preclude any ambiguity in the description of the invention.

The terms “short sidewall” and “tall sidewall” as used herein refer to two sidewalls, and one sidewall is bigger than the other one.

The term “cells” as used herein includes any cells from, but not limited to, microorganisms, plants and animals. Oocytes and animal embryos are currently preferred subjects for use with the present invention. Animal embryos may come from any desired mammalian sources including, but not limited to, humans; non-human primates, such as monkeys; laboratory mammals, such as rats, mice and hamsters; and farming livestock such as pigs, sheep, cows, goats and horses.

The term “microdrop” as used herein refers to a small drop of a culture medium containing cells that is formed by the cohesion of the culture medium being greater than the surface tension of the culture medium. The volume of each microdrop may be in the range of from 1 to 15 μl, preferably in the range of from 5 to 9 μl, more preferably in the range of from 3 to 4 μl, and most preferably in the range of from 1 to 2 μl.

The term “glass-like bead” as used herein refers to the microdrop of a culture medium that is rapidly frozen.

The term “embryo” as used herein refers to any zygote at an early stage of, but not limited to, morula, gastrula and blastocyst. The blastocyst is currently preferred.

The term “super-ovulated” as used herein refers to a physiological condition of the donor female animal treated with FSH so that a quantity of mature oocytes can be obtained at one time.

The term “embryo transfer” as used herein refers to the transfer of zygotes inseminated in vivo or in vitro to a recipient female.

The term “vitrification” as used herein refers to the phenomenon where solidification of a solution forms (glass formation) at a low temperature without ice crystal formation. This phenomenon can be regarded as an extreme increase of viscosity and requires either rapid cooling rates or the use of cryoprotectant solutions, which decrease ice crystal formation and increase viscosity at low temperatures.

All of the literature and publications recited in the context of the present disclosure are incorporated herein by reference.

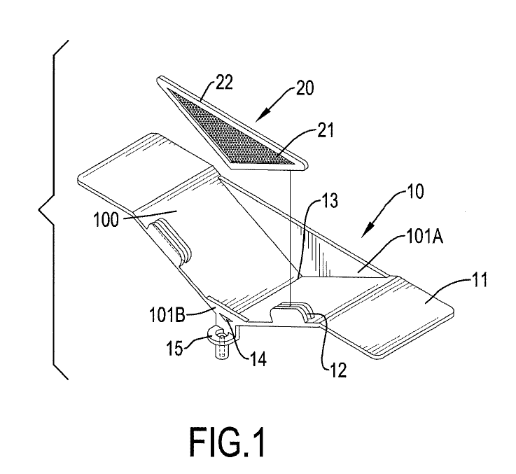

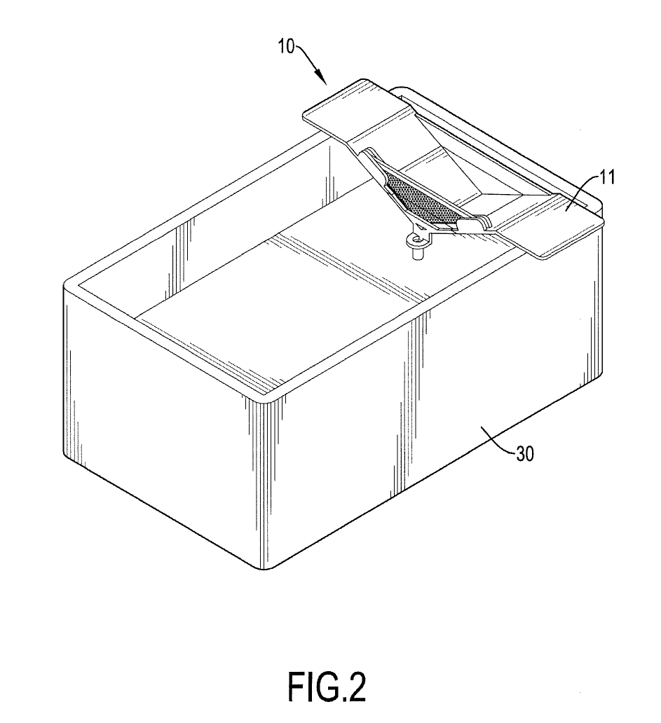

With reference to FIGS. 1 to 2, the vitrification apparatus for microdrop vitrification in accordance with the present invention is used for microdrop vitrification and vitrified microdrop thawing and comprises a tank (10), an optional filter (20), two wings (11) and an optional pressing stick.

The tank (10) is preferably V-shaped and has a depth (not numbered), a front (not numbered), a rear (not numbered), a bottom (not numbered), two side edges (not numbered), two top edges (not numbered), a front sidewall (101B), a rear sidewall (101A), two optional mounting slots (12), a lowest bottom (13), a drain (14) and a spout (15). The side edges are respectively at the front and rear of the tank (10). The sidewalls (101) are integrally formed respectively with and extend up from the side edges. The rear sidewall (101A) is formed at the rear edge and is tall, and the front sidewall (101B) is formed at the front edge and is short. The mounting slots (12) are respectively formed on the side edge having the short sidewall (101B). The lowest bottom (13) is defined in the bottom of the tank (10). The drain (14) is defined through the front sidewall (101B) at the bottom of the tank (10) and communicates with the lowest bottom (13). The spout (15) is connected to the bottom of the tank (10) and is aligned with the drain (14) to communicate with the lowest bottom (13).

The wings (11) are integrally formed respectively with and extend out from the top edges to mount the tank (10) on a container (30).

The filter (20) has a frame (22) and a mesh (21). The mesh (21) is mounted inside the frame (22). When the filter (20) is mounted in the mounting slots (12) on the tank (10), the front sidewall (101B) also holds the filter (20) in place. The mesh (21) is at most a 500 μm mesh, is more preferably a 300 μm mesh and is most preferably a 150 μl mesh.

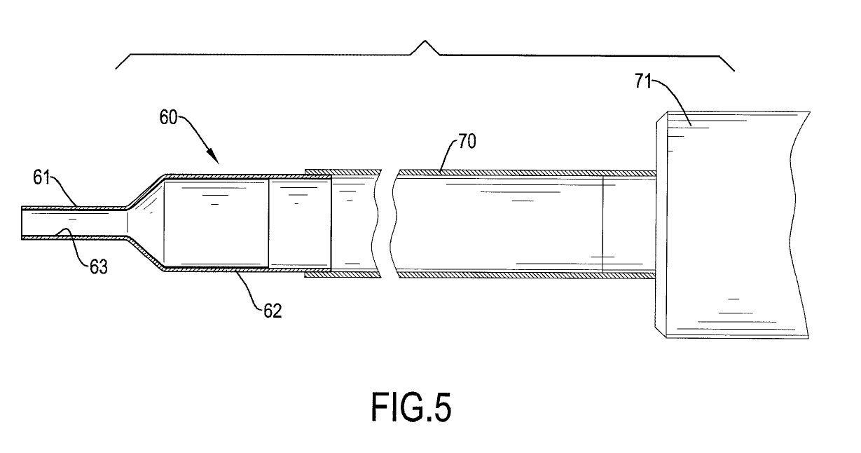

With reference to FIG. 5, the microdrop forming device comprises a modified capillary (60), a silicon tube (70) and a micro-syringe (71).

The modified capillary (60) has an inner surface (not numbered), an outer surface (not numbered), a capillary tip (61), a proximal end (62), a silicon membrane (63) and an optional calibration (not shown). The capillary tip (61) of the modified capillary (60) has a diameter (not numbered) and a specific volume (not numbered). The diameter is less than 0.8 mm and more preferably is in the range of 0.2 mm to 0.4 mm. The specific volume of the capillary tip (61) is 0.1 to 6 μl, more preferably is 0.1 to 5 μl, more preferably is 0.1 to 3 μl and most preferably is 1 to 2 μl. The silicon membrane (63) is formed on the inner surface of the modified capillary (60) by siliconization to provide a hydrophobic lining. The calibration of the outer surface of the modified capillary (60) is finished before use.

The silicon tube (70) has a distal end (not numbered) and a proximal end (not numbered). The distal end is attached to the proximal end (62) of the modified capillary (60).

The micro-syringe (71) is connected to the proximal end of the silicon tube (70) to draw a measured amount of culture medium into the capillary tip (61) of the modified capillary (60) to calibrate the capillary or to expel the medium to form a microdrop.

The method for microdrop vitrification of cells in accordance with the present invention comprises providing cells, culturing the cells, forming a microdrop containing cells, dropping the microdrop into liquid nitrogen, collecting the vitrified microdrops and storing the vitrified microdrops.

The cells may be provided from microorganisms, plants or animals. Cells from animals are oocytes or embryos, and the embryos are at the blastocyst stage.

The cells are cultured in a culture medium containing a cryoprotectant for a short period before vitrification.

The microdrops are formed from the culture medium containing the cells by using the microdrop-forming device. The microdrops are formed by drawing a specific amount of culture medium containing the cells into the capillary tip (61) of the modified capillary (60) with the micro-syringe (71) or mouth controlling.

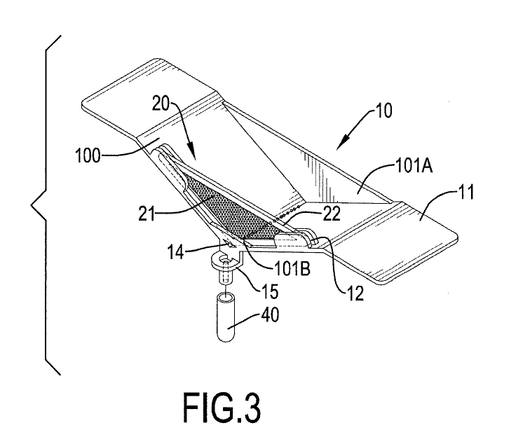

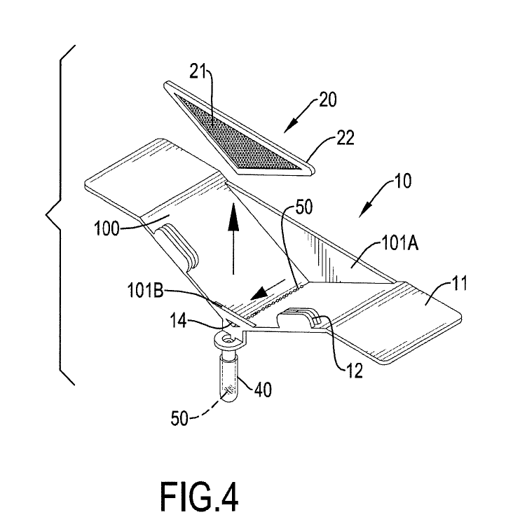

The microdrops are then dropped into the liquid nitrogen by flipping the capillary above the tank (10) of the vitrification apparatus. The tank (10) is submerged in the liquid nitrogen in the container preferably to approximately half the depth of the tank (10). The pressing stick is used to press the microdrop completely into the liquid nitrogen as soon as possible and is preferably cooled in the liquid nitrogen before use. With further reference to FIGS. 3 and 4, the liquid nitrogen vitrifies the microdrop and forms a glass-like bead (50).

When sufficient glass-like beads (50) are formed, a vial (40) is mounted on the spout (15). The filter (20) is removed from the mounting slots (12) to open the drain (14) in the tank (10), and the glass-like beads (50) are pushed to move pass through the drain (14) in the tank (10) and the spout (15) on the tank (10) and are finally collected in the vial (40).

The vial (40) containing the glass-like beads (50) is removed from the vitrification apparatus, and the glass-like beads (50) are stored at cryogenic temperatures until needed.

The method for thawing vitrified microdrops in accordance with the present invention comprises mounting the vitrification apparatus with the filter (20) on a container of liquid nitrogen, pouring the vitrified microdrops with cells into the tank (10), recovering the glass-like beads from the tank (10), and plunging them into a culture medium to thaw.

When the vitrification apparatus with the filter (20) is mounted on the container, the wings (11) are mounted on top edges of the container, and the tank (10) is partially submerged in liquid nitrogen in the container.

The glass-like beads (50) are poured from their storage container into the liquid nitrogen in the tank (10).

Finally, the glass-like beads are removed from the liquid nitrogen in the tank (10) with a liquid nitrogen cooled forceps and plunged into a culture medium to thaw.

The following examples are provided to assist people skilled in the art in performance of the invention and do not limit the scope of the invention previously described.

### EXAMPLES

### Example 1

### Preparation of a Microdrop-Forming Device

A conventional glass capillary was used to prepare the modified capillary (60). Preferably, the conventional capillary has a 0.8 mm inside diameter, a 1.1 mm outside diameter and a length of 100 mm. The middle of the capillary was heated, and ends of the capillary tube were pulled to stretch and reduce the diameter of the heated segment. The stretched capillary tube was removed from the heat and cut to obtain two intermediate modified capillaries.

To apply the silicone membrane (63) to the inner surface of the intermediate modified capillary, the proximal end of the intermediate modified capillary was connected to one end of a silicon tube (70), and the other end of the silicon tube (70) was connected to a 1 ml micro-syringe (71). The micro-syringe (71) drew liquid silicon (Sigmacot®) into the intermediate modified capillary and expelled the liquid silicon from the modified capillary (60). The modified capillary (60) was held upright to prevent liquid silicon on the inner surface from clogging the capillary tip (61) of the modified capillary (60). When the liquid silicon coating the inner surface dried, the modified capillary (60) was complete. Because the silicon membrane (63) is hydrophobic, liquid will discharge through the tip easily to form a droplet and will not adhere to the inner surface or remain inside the modified capillary (60).

The modified capillary (60) forms a microdrop by connecting to a silicon tube (70) that is connected to a micro-syringe (71) or controlled by a mouth. The micro-syringe (71) draws a medium with cells into the capillary tip (61) and then expelled out to form a microdrop. The outer surface of the modified capillary (60) may be precisely calibrated in advance by drawing the same volume size of the microdrop in the capillary tip (61).

### Example 2

### Microdrop Vitrification and Collection

2.1 Embryo Collection

2.1.1 Super-Ovulation

Healthy female goats with normal fertility were selected as female donors. The estrus cycles of the donors were synchronized with CIDR® (controlled internal drug release; CIDR, EAZI-BREEDTM, Australia) for 11 days. Beginning on the ninth day, the female goat donors were treated with follicle stimulating hormone from porcine pituitary (pFSH, KAWASAKI PHARMACEUTICAL CO, LTD., JAPAN) with gradually decreasing doses at 12-h intervals for six doses. The total dose for superovalation was pFSH. 20 A.U. and a dosage of 0.5 ml Estrumate (Cloptrostenol, 250 μg/ml synthesized prostaglandin F2α, Estrumate®, Schering-Plough, USA) was administered on day 9. The estrous does were mated with the bucks twice a day at an interval of 12 hours until the end of the estrous.

2.1.2 Embryo Collection

Embryos were collected by surgery on day 7 after the donor does showing estrus (day 0).

2.2 Preparation of Microdrop

2.2.1 Culture Media

TCM-199 culture medium containing 20% FCS

2.2.1.1 Freezing Media

TCM-199 culture medium containing 20% FCS, 10% ethylene glycol and 10% DMSO

TCM-199 culture medium containing 20% FCS, 16.5% ethylene glycol, 16.5% DMSO and 0.5 M sucrose

2.2.1.2 Thawing Media

TCM-199 culture medium containing 0.5 M sucrose and 20% FCS

TCM-199 culture medium containing 0.25 M sucrose and 20% FCS

TCM-199 culture medium containing 0.15 M sucrose and 20% FCS

TCM-199 culture medium containing 20% FCS

2.2.2 Process of Vitrification

The collected embryos were cultured in the TCM-199 containing 20% FCS for 5 minutes. Firstly, the embryos were transferred to the TCM-199 containing 20% FCS, 10% ethylene glycol and 10% DMSO for 45 seconds. Secondly, the embryos were transferred to the TCM-199 containing 20% FCS, 16.5% ethylene glycol, 16.5% DMSO and 0.5 M sucrose for a further 25 seconds.

The embryos cultured in the TCM-199 containing 20% FCS, 16.5% ethylene glycol, 16.5% DMSO and 0.5 M sucrose were immediately collected by the intermediate modified capillary as described above. A microdrop generated by the microdrop-forming device. Each microdrop (1 to 2 μl) contained 2 to 4 embryos. The microdrop was dropped into the vitrification apparatus that contained liquid nitrogen (LN₂) for vitrification and was frozen immediately to form a glass-like bead.

With reference to FIGS. 2 to 4, the glass-like beads (50) are lined up at the lowest bottom (13) of the tank (10) and are feasible to be visualized and collected by naked eyes. The glass-like beads are pushed to move forward by a pre-cooled forceps. A vial (40) was mounted on the spout (15) to collect the glass-like beads (50).

### Example 3

### Thawing the Glass-Like Beads and Culturing and Transferring the Embryos

3.1 Process of Thawing

The embryos were thawed before culturing and implantation. The glass-like beads containing the embryos were pouring into the filter (20)-mounted vitrification apparatus the same as for microdrop vitrification as described previously. A pre-cooled forceps was used to pick up the glass-like beads (50) from the lowest bottom (13), and then were plunged into the TCM-199 containing 0.5 M sucrose and 20% FCS at 38.5° C. for 5 minutes for thawing. Then the thawed embryos were transferred into the TCM-199 containing 0.25 M sucrose and 20% FCS for 5 minutes, then 0.15M sucrose and 20% FCS for 5 minutes. Finally, the embryos were moved to the TCM-199 culture medium containing 20% FCS for 5 minutes. Thawed embryos were observed for a few hours, and the survival rate of the embryos was recorded. The survived embryos were transferred directly to synchronized recipients. Pregnancy percentages and kidding rates were recorded.

3.2 Results

### Comparison Between a Conventional Freezing Method and the Present Invention.

| | | Transferred | Concep- | Embryo | |
|---|---|---|---|---|---|
| | Con- | embryo | tion | survival | |
| Spice | tainer | number (N) | rate (%) | rate (%) | Reference |
| Ovine | 0.25 ml | 50 | 72 | 50 | Baril et |
| | straw | | | | al. (2001) |
| | OPS | 28 | 71 | 61 | Isachenko et |
| | | | | | al. (2003) |
| | OPS | 10 | 50 | 35 | Papadopoulus |
| | | | | | et al. (2002) |
| Caprine | Straw | 59 | 56 | 37 | Cognie (1999) |
| | — | 31 | 81.3 | 64.5 | The present |
| | | | | | invention |

The improvement in the conception and survival rates of the present invention over those of conventional techniques are significant and are attributed to the increased freezing rate and reduced exposure to cryoprotectants associated with the current invention.

Directly subjecting the microdrops to liquid nitrogen in the present invention obviates the necessity for expensive equipment and materials associated with conventional vitrification techniques. Furthermore, virtually no additional complicate training or skills are required to operate the equipment and perform the methods in accordance with the present invention.

Because the cooling rate is improved, the method according to the present invention further reduces the likelihood of ice crystal formation and reduces the damage to the biological specimens caused by crystal formation.

Although the invention has been explained in relation to its preferred embodiment, many other possible modifications and variations can be made without departing from the spirit and scope of the invention as herein after claimed.

### LITERATURE REFERENCES AND PUBLICATIONS

1. J/R. Dobrinsky, 2002. Advancements in cryopreservation of domestic animal embryos. Theriogenology 57: 285-302.
2. Vajta G, Holm P, Kuwayama M, Booth P. J., Jacobsen H, Greve T, Callesen H., 1998. Open pulled straw (OPS) vitrification: A new way to reduce cryoinjuries of bovine ova and embryos. Molecular reproduction and development 51:53-58.
3. Baril G, Traldi A-L, Cognir B, Leboeuf B, Beckers J. F, Mermillod P., 2001. Successful transfer of vitrified goat embryo. Theriogenology 56:299-305.
4. Isachenko V., Alabart J. L., Dattena M., Nawroth F., Cappai P., Isachenko E., Cocero M. J., Olivera J., Roche A., Accardo C., Krivokharcheko A., Folch J., 2003. New technology for vitrification and field (microscope-free) warming and transfer of small ruminant embryos. Theriogenology 59: 1209-1218.
5. Papadopoulos S, Rizos D, Duffy P, Wade M, Quinn K, Boland M. P, Lonergan P., 2002. Embryo survival and recipient pregnancy rates after transfer of fresh or vitrified, in vivo or in vitro produced ovine blastocysts. Animal reproduction science 74: 35-44.
6. Cognie Y., 1999. State of the art in sheep-goat embryos transfer. Thenogenology 51: 105-116.
7. Rall W. F, Fahy G M., 1985. Ice-free cryopreservation of mouse embryos at −196° C. by vitrification. Nature Vol. 313.573-575.
8. Liebermann J, Nawroth F, Isachenko V, Isachenko E, Rahimi G, Tucker M. J., 2002. Potential importance of vitrification in reproduction medium. Biology of reproduction 67. 1671-1680.
9. Atabay E. C, Takahashi Y, Katagiri S, Nagano M, Koga, Kanai Y., 2004. Vitrification of bovine oocytes and its application to intergeneric somatic cell nucleus transfer. Thenogenology 61: 15-23.
10. Dinnyes A, Dai Y, Jiang S, Yang X., 2000. High development rates of vitrified bovine oocytes following parthenogenetic activation, in vitro fertilization, and somatic cell nuclear transfer. Biology of reproduction 63 (2): 513-518.

What is claimed is:

1. A microdrop forming device comprising:
a modified capillary having:
an inner surface,
a calibrated outer surface,
a capillary tip having a diameter less than 0.8 mm,
a proximal end, and
a silicon membrane formed on the inner surface,
a silicon tube having:
a distal end attached to the proximal end of the modified capillary, and
a proximal end, and
a micro-syringe connected to the proximal end of the silicon tube.

2. The microdrop forming device as claimed in claim 1, wherein the diameter of the capillary tip of the modified capillary is in the range of 0.2 mm to 0.4 mm.

3. The microdrop forming device as claimed in claim 2, wherein the modified capillary further has a specific volume in the range of 0.1 μl to 6 μl.

4. The microdrop forming device as claimed in claim 2, wherein the modified capillary further has a specific volume in the range of 0.1 μl to 3 μl.

5. The microdrop forming device as claimed in claim 2, wherein the modified capillary further has a specific volume in the range of 1 μl to 2 μl.
